(19) United States
(12) Patent Application Publication
(10) Pub. No.: US 2004/0034379 A1
(43) Pub. Date: Feb. 19, 2004

(54) Disposable ear canal cleaning appliance

(76) Inventor: Anthony K. Crespo, Alcoa, TN (US)

Correspondence Address: Susan F. Johnston, P.O. Box 4449, Johnson City, TN, 37602-4449, US

(21) Appl. No.: 10/639,372

(22) Filed: Aug. 12, 2003

Related U.S. Application Data
(63) Continuation-in-part of application No. 10/050,343, filed on Jan. 16, 2002.

(51) Int. Cl. A61F 9/00

(52) U.S. Cl. 606/162

(57) ABSTRACT

Disclosed herein is a disposable ear cleaning appliance for insertion into an ear canal. The appliance comprises an elongated stem and a bulbous cage extending from an end of the stem. The bulbous cage is defined by a plurality of bowed members extending from the end of the stem in an inwardly bowed attitude, to an uppermost point of intersection to form a bulbous cage small enough to be easily received in an ear canal.

### CROSS REFERENCES TO RELATED APPLICATIONS

This application is a continuation-in-part of prior-filed copending nonprovisional application Ser. No. 10/050,343, filed Jan. 16, 2002.

### FIELD OF INVENTION

This invention relates to appliances for cleaning foreign matter from the ear, particularly for removal of ear wax from the outer ear canal.

### BACKGROUND OF INVENTION

Possibly the most commonly known ear cleaner is the appliance marketed under the trade name Q-Tip. This product comprises a wad of cotton fibers bonded onto one end of an elongated stem or handle. These products, however, suffer from multiple problems including a tendency of a user to insert the appliance too far into the ear canal and the tendency of the cotton fibers to dislodge and remain within the ear canal.

In any ear cleaning appliance, it is desired that the portion of the appliance which enters the ear canal be readily capable of grasping any encountered foreign matter and retain the same on the appliance for removal of the foreign matter when the appliance is withdrawn from the ear canal.

Moreover, it is desirable that the appliance not be so rigid as to be capable of doing serious damage to the ear when inserted into the ear canal. Further, desirably, the ear cleaning appliance is disposable after one use, hence its cost of production should be minimal. For this latter reason, not only the cost of the raw materials employed in the appliance is of concern, but the cost of manufacturing the appliance is important.

### SUMMARY OF INVENTION

In accordance with a multi-piece embodiment of the present invention, there is provided a disposable ear cleaning appliance assembly comprising an elongated stem having a hollow cavity which opens outwardly from the upper end thereof, and a plurality, preferably four, flexible lengths of thermoplastic monofilament. These lengths of monofilament are joined at their outboard ends and extend therefrom to define a bulbous shape. At their respective outboard ends, the thermoplastic monofilaments are bonded by heat and pressure, for example, to form a blunt or flattened plate which serves to partially stiffen the individual monofilaments adjacent the bonded region and for a relatively short distance therefrom. Thus when the monofilaments are bent into a bulbous configuration by applying pressure against their respective inboard ends to gather the inboard ends into a bundle, the individual monofilaments are placed in compression and tend to retain their bulbous configuration. The accumulated inboard ends of the several monofilaments are gathered into a bundle in which the monofilaments lie in side by side in close packed relationship. This bundle of ends is inserted into the hollow cavity at the end of the stem and anchored therein as by sonic welding, adhesive or other suitable bonding technique.

In accordance with an alternative embodiment of the disposable ear cleaning appliance, there is provided an appliance formed from a thermoform plastic as a unitarily molded article. The thusly formed appliance comprises an elongate stem having a longitudinal axis, an upper end and a lower end, with a plurality of members having a bowed attitude and radiating longitudinally outward from positions about the upper end of the stem. Each of the bowed members extends from the upper stem end to an outermost perimeter, then upwardly inward to a point of intersection proximate the longitudinal axis, to define a bulbous cage at the upper end of the stem.

### BRIEF DESCRIPTION OF THE FIGURES

FIG. 1 is a representation of the multi-piece assembly embodiment of the appliance of the present invention;

FIG. 2 is an enlarged side plan view of one end of the multi-piece assembly appliance depicted in FIG. 1;

FIG. 3A is a schematic representation of a step in the manufacture of the multi-piece appliance of the present invention;

FIG. 3B is a schematic representation of a further step in the manufacture of the multi-piece appliance of the present invention;

FIG. 3C is a schematic representation of a still further step in the manufacture of the multi-piece appliance of the present invention;

FIG. 3D is a schematic representation of an even further step in the manufacture of the multi-piece appliance of the present invention; and

FIG. 4 is a representation of one use of the appliance of the present invention for cleaning the outer portion of an ear canal; and

FIG. 5 is a representation of the singe-piece embodiment of the present invention.

### DETAILED DESCRIPTION OF THE INVENTION

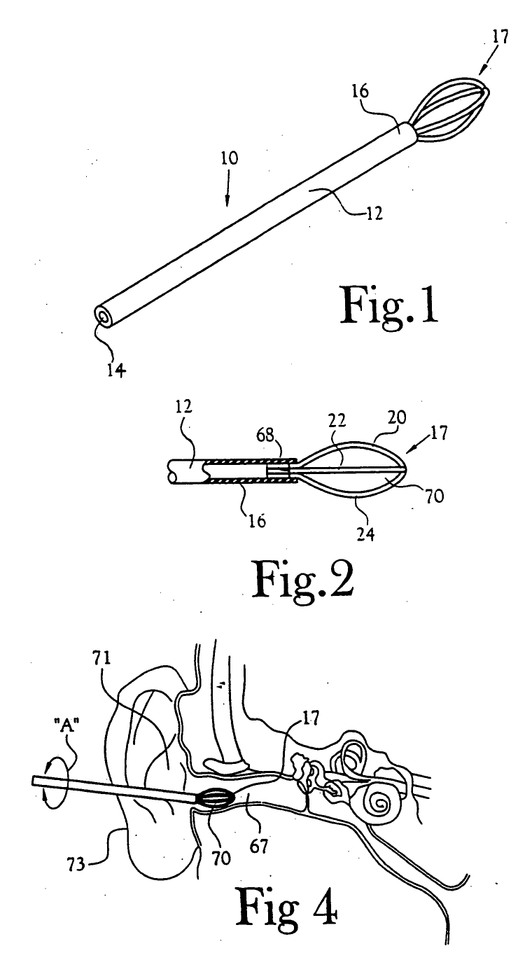

Referring initially to FIG. 1, a multi-piece embodiment of the appliance 10 of the present invention comprises an elongated stem 12 having a longitudinal axis, an upper end, and a lower end. The depicted embodiment of the stem 12 in FIG. 1 is in the form of a hollow tube 14 suitable to be grasped between the thumb and finger(s) of a user's hand. Within the upper end 16 of the hollow tube, there is secured a cleaning cage 17 adapted to enter the ear canal for the removal of foreign matter from the ear canal.

The stem 12 of the multi-piece embodiment of the present appliance comprises a hollow tube 14 about 6 cm long, and fabricated from a 15 gauge thermoplastic polymer, such as polypropylene, polyester or nylon. The outer diameter of the tube suitably is between about 2 and about 3 mm (millimeters) and the inner diameter is at least large enough to receive within an open end thereof a side-by-side closed packed bundle 68 (FIG. 2) of four or more ends of monofilament of between 18 and 25 gauge polymeric monofilament. The stem is substantially rigid, and readily grasped between the thumb and one or more fingers of the user's hand for insertion of the cage 17 into an ear canal and rotation of the stem and cage while the cage is within the ear canal.

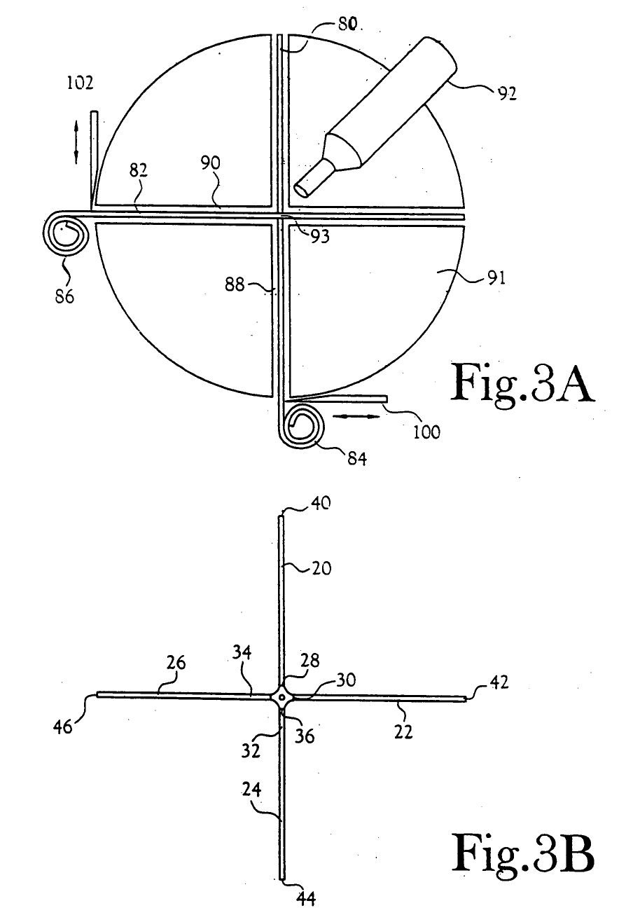

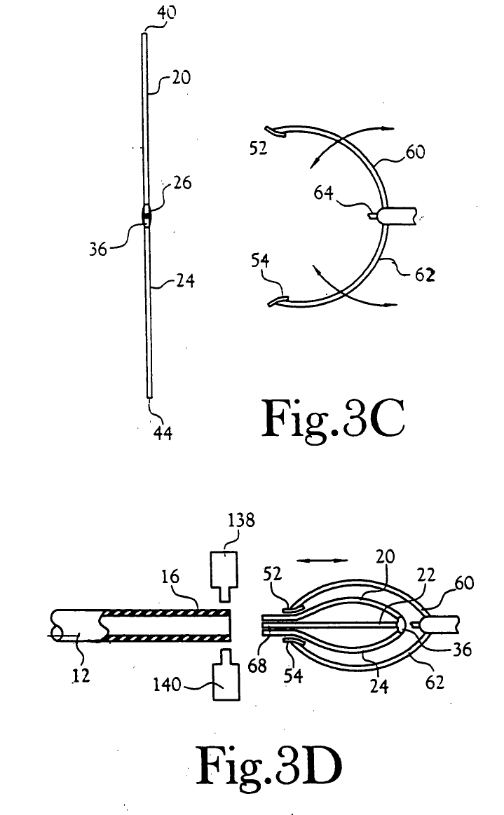

As depicted in FIG. 2, the cage 17 of the present multi-piece appliance comprises a plurality, four in a preferred embodiment, of lengths of monofilament 20, 22, 24 and 26. Only three of the lengths are visible in FIGS. 2 and 3D, but all four are depicted in FIG. 3B. Each length suitably is about 1.5 cm long. As best seen in FIGS. 2 and 3B, the outboard ends 28, 30, 32 and 34 of the lengths of monofilament are bonded together, as by sonic welding, to define a central mass 36 of molten and then solidified polymer which extends from the central mass into at least a portion of the length of each length of monofilament thereby rigidifying that portion of each monofilament which is adjacent the central mass 36. Thus, the lengths of monofilament are not only bonded to each other at their outboard ends, each length of monofilament includes a portion thereof which resists bending of the monofilament adjacent its outboard end by reason of the migrated and solidified thermoplastic thereon. By reason of this rigidity imparted to the outboard ends of the lengths of monofilaments, it has been found possible to contact the inboard ends 40, 42, 44 and 46 with respective pressure pads 50, 52 which are mounted on respective swing arms 60, 62 and bring the inboard ends of the lengths of monofilament together into a bundle 68 wherein the several inboard ends of the monofilaments lie in side-by-side closed packed relationship to on another and thereby cause each of the monofilaments to bow outwardly from one another to define the bulbous cage 17. It will be recognized that there is a like pressure pad and swing arm associated with each inboard end of monofilament even though only two such pressure pads and three such swing arms are depicted in the schematics of FIGS. 3C and 3D and that all four pressure pads and their respective swing arms function simultaneously to define the bundle of inboard ends of the lengths of monofilaments and, consequently, the bulbous nature of the cage.

The bundle 68 of inboard ends of the several lengths of monofilament is received within the hollow upper end 16 of the stem a distance of about 5 mm and bonded in place, as by sonic welding or other suitable attachment means, thereby securely anchoring the cage within the upper end 16 of the stem. By reason of the rigidity imparted to a portion of each length of monofilament adjacent its outboard end by the mass 36, once the inboard ends of the monofilaments are captured within the end of the stem, the individual monofilaments retain their bowed attitude. In turn, their collective bowed attitudes define the desired bulbous configuration of the cage.

Referring to FIG. 4, by reason of the structure of the cage described herein above, when the cage is inserted into an ear canal, each monofilament is free to bend slightly and thereby conform to whatever surface the cage may encounter within the ear canal, thereby permitting each portion of the irregular inner surface of the ear canal to be swept by rotating the stem of the appliance between the thumb and fingers of the user (See arrow “A” of FIG. 4). This action further serves to detach any foreign matter within the ear canal and collect the same on the surface of one or more of the lengths of monofilament, or between adjacent ones of the lengths of monofilaments, or within the interior region 10 of the bulbous cage of the appliance. Upon withdrawal of the appliance from the ear canal, the captured foreign matter is likewise withdrawn from the ear canal. Moreover, the flexible nature of the individual lengths of monofilaments of the cleaning cage permit the cage to also be used to clean the outer surfaces 71 of the ear lobe 73 in like manner.

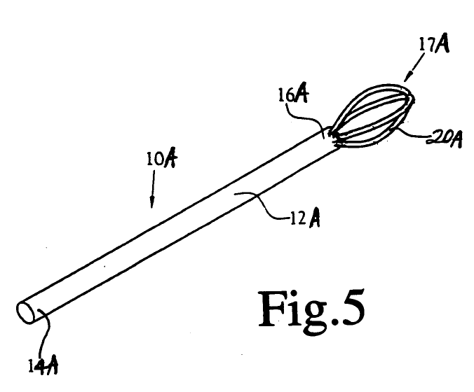

In both the multi-piece and single-piece embodiments of the present invention, the bulbous cage has a diameter across the outermost perimeter of the cage, transverse to the longitudinal axis of the stem, smaller than the diameter across the ear canal, as shown in FIG. 4. Further, it is preferred that the width of each of the monofilaments of the multi-piece embodiment 10 and corresponding bowed members of the single-piece embodiment 10A in FIG. 5 is between 0.25 to 0.10 of the transverse diameter across the outermost perimeter of the cage so as to impart both scraping and capturing capabilities to the cage.

Referring to FIGS. 3A-3D, in one embodiment of a method for the manufacture the multi-piece appliance assembly according to the present invention, first and second monofilaments 80 and 82, respectively, are fed from coiled rolls 84 and 86, respectively, into perpendicularly intersecting grooves 88, 90 in a die 91 such that the monofilaments cross over one another at the intersecting midpoints 93 of the grooves. Each of the monofilaments is severed from its respective coil at its point of entry into a groove as by reciprocating knives 100, 102. Thereupon, at the intersection of the monofilaments, they are bonded to one another by a sonic sealer 92 which applies pressure against the intersection of the filaments while heating the same to at least the fusion point of the thermoplastic. In the present instance, the pressure applied by the sonic sealer to the intersection of the monofilaments in the course of heating the monofilaments, substantially flattens the monofilaments causing the thermoplastic material to flow laterally away from the intersection of the monofilaments and thereby produce a substantial mass 36 (FIG. 3B) of molten thermoplastic material at the intersection. Under the influence of the applied pressure, this molten mass spreads laterally in a direction away from the mass and along the length of each of the monofilaments and when the heat is removed, the mass cools and solidifies. This action results in a stiffening of each of the monofilaments in the region thereof adjacent the central mass of solidified thermoplastic. As noted hereinabove, this mass of solidified thermoplastic is effective in limiting the degree of bowing of the lengths of monofilament into a bulbous head. Further, the bonding of the two initial lengths of monofilament 80 and 82 at their intersection, there are defined the aforesaid four lengths of monofilaments 20, 22, 24 and 26, each having its outboard end bonded to the outboard end of each other filament. Notably, as 20 seen in FIG. 3B, the four monofilaments extend laterally away from their juncture of joinder in respective directions that are oriented at 90 degrees intervals about their juncture of joinder.

After bonding of the intersection of the monofilaments, the interim product depicted in FIG. 3B is formed into a bulbous configuration by contacting the outboard ends 28, 30, 32 and 34 of the monofilaments with pressure pads which are mounted on swing arms and moving the length of monofilament to bring the outboard ends of the monofilaments together into side-by-side closely packed relationship as a bundle 68, as shown in FIGS. 3C and 3D. Thereafter, the bundle of inboard ends of the monofilaments is inserted into the open upper end of the hollow tube and bonded within the tube as by sonic welders 138, 140.

Other methods of manufacture of the multi-piece assembly may be employed. For example, the grooves depicted in FIG. 3A may be cylindrical holes drilled through the die 91. Still further, the size of the solidified mass of thermoplastic material, and the extent of lateral flow thereof along each of the monofilaments, may be adjusted through selection of the size of the horn of the sonic sealer and/or the amount of pressure applied to the molten thermoplastic at the intersection of the monofilaments.

The present invention further provides for a single-piece embodiment of the ear cleaning appliance formed as a unitarily molded plastic object. The most suitable manufacturing method for forming the singe-piece embodiment from the above described thermoform plastic is injection molding using a mold adapted to provide an apparatus having the characteristics described herein below.

As shown in FIG. 5, the structural relationship of the various elements of the single-piece embodiment of the ear cleaning appliance 10A is very similar to that of the multi-piece assembly 10 shown in FIG. 1 and described above, except that the cleaning cage and stem are unitarily formed so that the bowed members defining the cage are actual extensions of the upper end of the stem instead of being a separate piece inserted into the stem. Thus, the members can either extend from the enclosed end surface or from the cylindrical side surface at the end of the solid stem.

More specifically with reference to FIG. 5, in the single-piece appliance 10A, the stem 12A may be either hollow or solid, preferably solid. The bulbous cage 17A is formed on the upper end 16A of the stem by a plurality of slender members 20A radiating directly from points about the upper end 16A and extending, in a bowed attitude, to an outermost perimeter then extending, still in the bowed attitude, upwardly inward to a point 93A proximate the longitudinal axis. The point 93A may be a mere point of intersection of the members or else a plate 36A corresponding to the substantial mass 36 of the multi-piece appliance. Regardless, the upper end of the cage should be rounded or blunt so as to prevent injury to the ear. The cleaning cage should have no sharp edges.

A suitable number of bowed members 20A defining the cage 17A in the single-piece embodiment is any number providing a combination of scraping and capturing debris from the ear canal, preferably at least four, with between four and eight members being more preferable.

Further modifications of the present invention, in both the multi-piece and the single piece embodiments, include the presence of another useful head at the lower end of the stem. For example, a second bulbous cage of same or different size as the cage at the upper end of the stem, an absorbent swab, a pad, and the like may be incorporated into or attached to the lower end of the stem to improve or complement the ear cleaning application.

Whereas the dimensions of the stem and the cage are set forth herein as preferred, it will be understood that variations in these dimensions which do not materially alter the functioning of the appliance may be employed. The invention has been described in detail with particular reference to preferred embodiments thereof, but it will be understood that variations and modifications can be effected within the spirit and scope of the invention.

What is claimed:

1. An ear cleaning appliance comprising:
an elongate stem having a longitudinal axis, an upper end, and a lower end; and
a bulbous cage extending upwardly from the upper end of said stem, said bulbous cage defined by a plurality of bowed members extending upwardly to a point proximate the longitudinal axis.

2. The ear cleaning appliance according to claim 1 wherein said bulbous cages has a transverse diameter smaller than an ear canal.

3. The ear cleaning appliance according to claim 1 wherein said plurality of bowed members is between four to eight members.

4. An ear cleaning appliance comprising:
an elongate stem having a longitudinal axis, an upper end, a lower end, a plurality of members having a width, each of said members radiating outwardly from positions about said upper end, with an inwardly bowed attitude, to an outermost perimeter and extending upwardly inward, at the inwardly bowed attitude, to a point of intersection proximately on the longitudinal axis, thus forming a bulbous cage at said upper end having a diameter across the outermost perimeter smaller than the diameter of an ear canal,
wherein said appliance is a unitarily molded article formed from a thermoform plastic.

5. The ear cleaning appliance according to claim 4 wherein said plastic is selected from the group consisting of polyethylene, nylon, and polypropylene.

6. The ear cleaning appliance according to claim 4 wherein said plastic is polypropylene.

7. The ear cleaning appliance according to claim 4 wherein said plurality of members is between four to eight members.

8. The ear cleaning appliance according to claim 4 wherein said point of intersection is on a plate having a center transected by the longitudinal axis.

9. The ear cleaning appliance according to claim 8 wherein said plate has a transverse diameter between about 1 to about 4 times the width of each of said members.

10. The ear cleaning appliance according to claim 4 wherein the width of each of said members is between about 0.25 to 0.1 times the diameter across the outermost perimeter of said bulbous cage.

11. A process of forming a disposable ear cleaning appliance comprising:
molding a thermoform plastic using a die sufficiently adapted to provide a single-piece appliance having an elongate stem having a longitudinal axis, an upper end, a lower end, a plurality of members having a width, each of said members radiating outwardly from positions about said upper end, with an inwardly bowed attitude, to an outermost perimeter and extending upwardly inward, at the inwardly bowed attitude, to a point proximate the longitudinal axis, thus forming a bulbous cage having a diameter across the outermost perimeter smaller than the diameter of an ear canal.

12. The process of claim 4 wherein said molding is conducted using an injection molding method.

13. The process of claim 11 wherein said thermoform plastic is selected from the group consisting of polyethylene, nylon, and polypropylene.
